(19) United States
(12) Patent Application Publication
(10) Pub. No.: US 2014/0030236 A1
(43) Pub. Date: Jan. 30, 2014

(54) DERIVATION AND MATURATION OF SYNTHETIC AND CONTRACTILE VASCULAR SMOOTH MUSCLE CELLS FROM HUMAN PLURIPOTENT STEM CELLS

(71) Applicant: The Johns Hopkins University, Baltimore, MD (US)

(72) Inventors: Maureen Wanjare, Baltimore, MD (US); Sharon Gerecht, Baltimore, MD (US)

(73) Assignee: The Johns Hopkins University, Baltimore, MD (US)

(21) Appl. No.: 14/044,006

(22) Filed: Oct. 2, 2013

Related U.S. Application Data
(63) Continuation-in-part of application No. 13/581,341, filed on Aug. 27, 2012.
(60) Provisional application No. 61/308,014, filed on Feb. 25, 2010.

(51) Int. Cl. C12N 5/077

(52) U.S. Cl. CPC C12N 5/0661
USPC 424/93.7; 435/377

(57) ABSTRACT

Embryonic vascular smooth muscle cells (vSMCs) have a synthetic phenotype (Syn-vSMC), but in adults, they commit to the mature contractile phenotype (Con-vSMC). Con-vSMCs differ from Syn-vSMC derivatives in condensed morphology, prominent filamentous cytoskeleton proteins, elastin production and assembly elastin, low proliferation, numerous active caveolae, enlarged endoplasmic reticulum, ample stress fibers and bundles, as well as high contractility. The human pluripotent stem cell-derivatives can differentiate into a desired phenotype. Differentiation can be controlled by appropriate concentrations of relevant factors. Growth in high serum with platelet-derived growth factor-BB (PDGF-BB) and transforming growth factor-β1 induces the Syn-SMC phenotype with increased extracellular matrix protein expression and reduced expression of contractile proteins. Serum starvation and PDGF-BB deprivation causes maturation towards the Con-vSMC phenotype. When transplanted subcutaneously into nude mice, the human Con-vSMCs aligned next to the host's growing functional vasculature, with occasional circumferential wrapping and vascular tube narrowing.

This application is a continuation-in-part application of, and claims the benefit of, copending U.S. patent application Ser. No. 13/581,341, filed Aug. 27, 2012, which is a National Stage of PCT/US2011/026294 filed Feb. 25, 2011, which claims the benefit of the filing date of U.S. Provisional Patent Application 61/308,014, filed Feb. 25, 2010, each of which is incorporated by reference in their entirety herein.

This invention was supported by National Institutes of Health grant R01HL107938; the U.S. government may have certain rights to this invention.

### BACKGROUND

1. Area of the Art

The present invention is in the area of tissue differentiation from stem cells and more particularly discloses a process for differentiating two different phenotypes vascular smooth muscle cells from pluripotent stem cells

2. Description of the Background

The stabilization of blood vessels occurs by extracellular matrix (ECM) formation, as well as through the recruitment of mural cells, which include vascular smooth muscle cells (vSMCs) and pericytes. While pericytes are found in the microvasculature, such as in capillaries, vSMCs surround larger vessels like arteries and veins. During angiogenesis, endothelial cells (ECs) proliferate; connect to preexisting blood vessels; and, through lumen formation, develop endothelial tubules (a process known as intussusception) (1). After the formation of the nascent tubes composed of ECs, surrounding undifferentiated mesenchymal cells get recruited and become differentiated into proliferating vSMCs, which are needed to stabilize the formed tubules (2, 3). Platelet-derived growth factor (PDGF-BB) (4, 5) and transforming growth factor (TGF-β1) (6, 7) act as signaling cues for the recruitment and differentiation of vSMCs. Research has suggested that vSMCs become quiescent after birth, taking on the contractile phenotype found in adult vessels (10, 11).

During neovascularization in the embryo (12) or during vessel development, vSMCs have a synthetic phenotype, which is characterized by high proliferation, migration, and ECM protein production (13). In adult blood vessels, vSMCs play an important role in vessel stabilization; therefore, they commit to the mature contractile phenotype, characterized by low proliferation, expression of contractile proteins—namely, smooth muscle myosin heavy chain (SMMHC) and elastin—and low synthetic activity (13).

Adult vSMCs wrap around the vessel layer of ECs and contract to regulate and maintain blood vessel diameter in order to counteract the pulsatile blood pressure generated by the heart (14). Remarkably, vSMCs do not stay in a particular terminally differentiated state. Instead, they exhibit plasticity—they can reversibly take on either a contractile or a synthetic phenotype (13).

Pluripotent stem cells (PSCs), including human embryonic stem cells (hESCs) and induced pluripotent stem cells (iPSCs), serve as a reliable source for vSMCs because they can self-renew and proliferate. Pluripotent stem cells first differentiate into the mesoderm (15) and later into the vascular lineages, including vSMCs (16, 17). Collagen IV, (16) retinoic acid (19-21), and the growth factors PDGF-BB5, (16, 21-23) and TGF-β (17) have been implicated in the inducement of vSMC differentiation. Vascular SMCs have previously been derived from human iPSCs from skin fibroblasts (24) and human aortic smooth muscle (25). To the best of our knowledge, no study has demonstrated the regulation of both contractile proteins, SMMHC and elastin, in the course of the differentiation and maturation of vSMCs from PSCs.

We previously found that the derivation of vascular smooth-muscle-like cells (SMLCs) from hESCs could be achieved using monolayer cultures supplemented with PDGF-BB and TGF-β1.5, (26). The parent of this application extended that work and the present disclosure shows that hPSC-derived SMLCs can be guided to acquire either a synthetic or contractile phenotype.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows that SMLCs take on a synthetic phenotype. On day 12 of culturing SMLCs, they were moved to differentiation medium containing 10% serum with PDGF-BB and TGF-β1 for an additional 18 days. FIG. 1A: Quantitative RT-PCR revealed no significant changes in the expression of SMA and SM22 and showed a decrease in calponin and SMMHC between SMLCs and Syn-vSMCs. FIG. 1B: Western blot analysis confirmed the protein expression of SMA, calponin, and SM22 in SMLCs, Syn-vSMCs, and aortic vSMCs. FIGS. 1C-D demonstrate increased expression of ECM proteins, collagen I, and fibronectin and a decrease in the expression of elastin in Syn-vSMCs compared to SMLCs. FIG. 1E: Upregulation in the expression of MT-1 MMP, MMP1, and MMP2 in Syn-vSMCs. Scale bars are 100 μm. *p<0.05, **p<0.01, and ***p<0.001.

FIG. 2 shows that serum starvation and PDGF deprivation induce contractile phenotype maturation. FIG. 2A shows Syn-vSMCs and FIG. 2B shows SMLCs cultured for 6 additional days in media containing 10% serum with PDGF-BB and TGF-β1, 10% serum with TGF-β1, and 0.5% with TGF-β1; they were analyzed for the mRNA expression of relevant cytoskeleton and ECM genes. FIG. 2C: Immunofluorescence analysis of mSMLCs demonstrates the expression and organization of various cytoskeleton proteins, including calponin, SMA, SM22, phalloidin, and SMMHC, as well as the expression of elastin. Scale bars are 100 μm. *p<0.05, **p<0.01, and ***p<0.001.

FIG. 3 shows that mature SMLCs acquire a contractile phenotype, with or without the addition of Transforming Growth Factor beta 1 (TGF-β1. H9-hESC-derived mSMLCs were cultured in medium containing 0.5% serum, with and without TGF β1, for (FIG. 3A) an additional 6 days and (FIG. 3B) an additional 12 days and analyzed for the mRNA expression of relevant cytoskeleton and ECM genes. FIG. 3C: Comparison of the mRNA expression of pathway regulators during the stages of differentiation and maturation. *p<0.05, **p<0.01, and ***p<0.001.

FIG. 4 shows characterization of Con-vSMCs and Syn-vSMCs. FIG. 4A: The two phenotypes were characterized for morphology, using light microscopy (LM), and the organization of various cytoskeleton proteins, including calponin, αSMA, SMMHC, SM22, and phalloidin, as well as the expression of elastin, all using immunofluorescence staining. FIG. 4B: Con-vSMCs cultured for 6 days were analyzed for elastin assembly using immunofluorescence staining. FIG. 4C: Proliferative cells were detected using immunofluorescence staining of Ki-67 in both cell phenotypes. Scale bars are 100 μm. (D) TEM analysis showed (i) caveolae (arrowheads) with occasional fusion (asterisk); (ii) ER; and (iii) stress fibers (arrows). ER=endoplasmic reticulum.

FIG. 5 shows the functionality of hPSC derivatives. FIG. 5A: Contractility rates of SMLCs (n=9), Syn-vSMCs (n=12), Con-vSMCs (n=9), and human aortic vSMCs (n=12). FIG. 5B: Subcutaneously transplanted Con-vSMCs migrated to newly formed blood vessels within the Matrigel, as indicated by representative (i) confocal images; and (ii-iii) light microscopy images of human elastin-stained sections. Scale bars are 50 μm. FIG. 5C: Occasionally, Con-vSMCs were found wrapping around the functional vasculature. The high-magnification inset shows the boxed area. FIG. 5D: (i) 3D reconstruction of confocal microscopy images revealed an event of tube narrowing of small vessels by the transplanted Con-vSMCs further demonstrated by (ii) measurements of adjusted areas within the vessel with and without Con-vSMC wrapping. Scale bars are 20 μm. For confocal images, Con-vSMCs in red [some indicated with white arrows]; mouse endothelial cells in green; and nuclei in blue). *p<0.05, **p<0.01, and ***p<0.001.

FIG. 6 shows SMLC compared to human aortic vSMCs; quantitative RT-PCR analysis shows the relative mRNA expression of the relevant cytoskeleton and ECM genes of the differentiated SMLCs compared to human aortic vSMCs.

FIG. 7 shows SMLCs differentiation. SMLCs were cultured (from day 12) in differentiation media containing 10% serum with PDGF-BB and TGF-β1, with TGF-β1 only, or without any growth factors.

FIG. 8 shows maturation of hiPSC lines. Mature SMLCs derived from (FIG. 8A) BC1 and (FIG. 8B) MR31 were cultured in media containing 0.5% serum, with and without TGF-β1, for an additional 12 days and analyzed for the mRNA expression of relevant cytoskeleton and ECM genes.

FIG. 9 shows Con-vSMCs compared to human aortic vSMCs. Quantitative RT-PCR analysis shows the relative mRNA expression of the relevant cytoskeleton and ECM of the differentiated SMLCs compared to human aortic vSMCs.

FIG. 10 shows pathways in BC1 hiPSCs. FIG. 10A shows a comparison of mRNA expression of pathway regulators during the stages of the differentiation and maturation of BC1. FIG. 10B shows quantitative RT-PCR analysis of the relative mRNA expression of SRF and myocardin BC1-hiPSC-SMLCs compared to H9-hESCs-SMLCs.

FIG. 11 shows TEM analysis and functionality of hPSC derivatives. FIG. 11A: High resolution imaging of organelles in Syn-vSMCs and Con-vSMC showing (i) caviolae (arrowheads) ER, and stress fibers (arrows); (ii) actin bundles (asterisks). ER=endoplasmic reticulum; N=nucleus. FIG. 11B: Subcutaneously transplanted Syn-vSMCs migrated to newly formed blood vessels within the Matrigel, as indicated by representative confocal images. Syn-vSMCs in red; mouse endothelial cells in green; and nuclei in blue).

FIG. 12 is a schematic for culture protocol to induce synthetic and contractile phenotypes from SMLCs. An adherent culture of hPSCs in media containing high (10%) serum concentration and PDGF-BB and TGF-β1 induces their differentiation into SMLCs within 12 days (5). Long-term differentiation of SMLCs in media containing 10% serum, as well as PDGF-BB and TGF-β1, induces their maturation into Syn-vSMCs within 18 days. Syn-vSMCs could be expanded in their synthetic phenotype using high serum media but not acquire upregulation in the expression of SMMHC and elastin when cultured in low serum media. Culturing SMLCs in serum starvation (0.5%) with TGF-β1 supplementation for an additional 6 days guided contractile mSMLCs. Downregulation in the expression of SMMHC and elastin was achieved when culturing mSMLCs in media containing high serum concentration. An additional 12 days of continuous serum starvation, with and without TGF-β1 supplementation, induced upregulation of SMMHC and elastin, i.e., caused the mSMLCs to mature into Con-vSMCs. Con-vSMCs could maintain high expression levels of SMMHC and elastin in low-serum media and down-regulate the contractile proteins when cultured in high-serum media.

### DETAILED DESCRIPTION OF THE INVENTION

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide a method for controlling the differentiation of pluripotent stem cells into synthetic or contractile vascular smooth muscle cells (vSMCs).

Previous studies by the present inventors demonstrated that we could derive vascular lineages from hESCs by administering angiogenic growth factors using a two-dimensional (2D) monolayer differentiation protocol or by isolating vascular progenitor cells or CD34⁺ cells from ten-day-old EBs, followed by selective induction into either endothelial-like cells (using vascular endothelial growth factor [VEGF]) or SMLCs (using PDGF-BB) (27, 31). More recently, building on these initial studies, the inventors established a simple step-wise differentiation protocol that cultured hPSCs in monolayers and supplemented them with PDGF-BB and TGF-β1, resulting in highly purified cultures of SMLCs (5). These SMLCs were more than 98% positive for SMA, calponin, and SM22 and about 50% positive for SMMHC. They produced collagen and fibronectin, and they contracted in response to carbachol. Further in vitro tubulogenesis assays revealed that these hPSC-derived SMLCs interacted with human endothelial progenitor cells to support and augment the formation of cord-like structures (5). The present invention is directed to using these SMLCs to make the synthetic versus contractile phenotype decision.

Synthetic-vSMCs produce ECM proteins, such as collagen and fibronectin, as well as MMP proteins, in order to aid in cell migration (34). Long-term (up to 30-day) cultures of the differentiated SMLCs in high serum with PDGF-BB and TGF-β1 result in maturation towards a synthetic phenotype, reducing the expression of contractile proteins and increasing the expression of ECM proteins, collagen, fibronectin, and MMPs. Indeed, both of these growth factors were suggested in early stages of differentiation (5, 27, 31). Attempts to eliminate only PDGF-BB or both growth factors from the culture media somewhat increase synthetic phenotype characteristics (i.e., SMMHC and elastin expression), suggesting that this strategy may be useful for guiding the contractile phenotype. Nonetheless, after their long-term exposure to PDGF-BB and TGF-β1, these Syn-vSMCs seemed unable to acquire a contractile phenotype when deprived of serum and growth factor, suggesting a terminal synthetic phenotype.

To mimic the native state of vSMCs in vessels, requires a switch between a quiescent and contractile state (31) Quiescence is marked by the reduction of the proliferative capacity of a cell. Vascular SMCs in vessel walls replicate at the low frequency of 0.047% per day (35). In this low proliferative state, the vSMC becomes committed to its contractile function (13, 32). Growth factors, as well as fetal calf serum, drive the proliferative capacity of vSMCs (34). However, it is still not known how the proliferative state of native vSMCs becomes suppressed. Moreover, it has been suggested that PDGF-BB interferes with vSMC maturation (33-36). SMMHC has a high specificity for SMCs and is also considered a mature marker indicating a contractile phenotype (13). The ECM protein elastin also gets expressed in the contractile state (37, 38). In adult vSMCs, elastin acts as an autocrine regulator and also determines mechanical responsiveness (39). Indeed, when SMLCs were matured in media containing low concentrations of serum and supplemented with TGF-β1, upregulation of SMMHC and elastin in the mSMLCs is observed. These mSMLCs seem to retain plasticity, as indicated by downregulation of the contractile proteins SMMHC and elastin when differentiated in media containing high concentrations of serum.

Continued quiescence of mSMLCs in media containing low concentrations of serum and supplemented with or without TGF-β1 induced additional upregulation in the expression of contractile proteins. These Con-vSMCs maintained their contractile phenotype when cultured in low serum conditions; they exhibited plasticity with the downregulated expression of contractile protein when cultured in high serum concentrations.

Myocardin, a potent SRF coactivator expressed exclusively in vSMCs and cardiomyocytes (40) reportedly promoted SMC differentiation through transcriptional stimulation of SRF-dependent smooth muscle genes, including SMMHC (41, 42). A recent study demonstrated that myocardin^{-/-} mouse ESCs differentiate to vSMCs, suggesting the dispensability of myocardin for the development of vascular SMCs (43). In support of this observation, we report that deprivation of TGFβ1 seems to affect the activation of the different pathways, although upregulation of contractile proteins was observed. Overall, the present inventors' data using hPSCs shows that upregulating the myocardin pathway was not necessarily associated with the contractile state of the differentiating vSMCs. Finally, Both YAP1 and SMAD3 have been suggested as regulators important for inducing the synthetic phenotype in vSMCs (44, 45). Present data show that these also get upregulated during the synthetic phenotype maturation of hPSC derivatives. Here as well, deprivation of TGFβ1 affects the activation of these pathways in contractile maturation.

Comparing Con-vSMC and Syn-vSMC derivatives, it was observed that both acquire a more spindle-shaped morphology than SMLCs. More prominent filamentous organization of the various cytoskeleton proteins was found in Con-vSMC than in Syn-vSMC derivatives. Interestingly, both cell derivatives showed increases in contractility: Syn-vSMCs showed some increased contractility, which may be attributed to the needed optimization of the culture period and to cell confluence; Con-vSMCs exhibit a rather greater increase in contractility than human aortic vSMCs, most likely due to higher SMMHC expression. Reducing the serum concentrations in media of SMLCs markedly decreased the proliferation rates of the cells and was accompanied by an increase in the contractile phenotype. Indeed, the Con-vSMC phenotype was marked by a reduced proliferative capacity, unlike the Syn-vSMC phenotype, which exhibited a high proliferative capacity. Finally, high-resolution analysis further revealed profound differences previously observed between the two phenotypes (46). Unlike Syn-vSMCs, Con-vSMCs exhibited numerous and active caveolae with enlarged ER and abundant stress fibers and bundles, underlining the distinctive shift between two major differentiated states with distinct morphological and functional properties.

Researchers envision human iPSCs—which can be derived directly from a patient, thereby reducing the risk of immunogenicity upon transplantation—as dramatically revolutionizing cell-based therapies for regenerative medicine. Since Takahashi and Yamanaka's pioneering discovery (47), hiPSC technology has evolved rapidly. While the hiPSC technologies initially reported have several obvious shortcomings, many of these have recently been overcome. This study tested MR31—a hiPSC clone derived from the IMR90 line, which was derived from normal fetal lung fibroblasts using a lentivirus to deliver three reprogramming factors (Oct-4, Sox2 and Klf4)²⁵—and BC1, which was induced using CD34+ blood cells from bone marrow using plasmids encoding all four reprogramming factors (28, 29). We have shown that hiPSCs respond to the differentiation protocol similarly to hESCs and can mature into the synthetic and contractile phenotypes of vSMCs. The mSMLCs derived from all the hPSCs examined exhibited comparable expression levels of both SMMHC and elastin. We observed some differences during their long-term exposure to serum starvation with and without TGF-β1. Specifically, when culturing mSMLCs derived from MR31 in a low concentration of serum, with or without TGF-β1, we detected upregulated elastin expression and downregulated expression of SMMHCs. The derivation of vSMCs from the BC1 line, an integration-free induced PSC line (28, 29) offers a practical approach for using this clinically relevant technology for vascular regeneration. Thus, it seems apparent that hiPSCs have immense potential for providing effective treatments or cures for vascular diseases, which warrants further investigations and improvements.

Previous studies suggested that vSMCs wrap circumferentially rather than longitudinally around blood vessels (48, 49). Some have suggested that this wrapping improves the mechanical properties (31, 50) of the vessel wall while also managing proper vasoactive activity (31). In early studies, the inventors demonstrated the contribution of vSMC-derivatives of a synthetic nature to growing vasculature (5, 31). Studies related to the present invention tested whether the Con-vSMCs could still migrate towards a growing vessel, as well as begin wrapping. Utilizing a subcutaneous transplantation model assay, it has been shown that Con-vSMCs encapsulated in Matrigel plugs migrate to sites near newly grown functional vasculature where they produce elastin that stabilizes those vasculatures. Moreover, the Con-vSMCs were sometimes found wrapping and even narrowing the host vessels. Such Con-vSMC provide opportunities to use such derivatives to enhance the stabilization and maturation of new blood vessels in regenerating tissues.

In summary, the present invention provides a method for manipulating fate decisions in vascular smooth muscle phenotypes during the differentiation of hPSCs. By monitoring the expression of SMMHC and elastin, the possibility of generating synthetic or contractile phenotypes from different hPSC lines with appropriate concentrations of factors known to control these developmental steps in the early embryo and in adulthood is demonstarted. This highlights the importance of designing stage-specific differentiation strategies that follow key developmental steps to exploit cellular plasticity for vSMC phenotypic decisions. Finally, contractile hPSC-vSMCs derived from the integration-free hiPSC line BC1 may prove useful for regenerative therapy involving blood vessel differentiation and stabilization.

In embodiments, the present invention is a method for differentiating undifferentiated mammalian vascular smooth muscle-like cells (SMLCs) into vascular smooth muscle-like cells (SMLCs) with a contractile (Con-vSMLCs) phenotype in vitro, that includes the steps of (1) exposing the SMLCs to serum starvation with TGF β1 to differentiate the SMLCs into mature SMLCs(mSMLCs); and (2) treating the mSMLCs to continued serum starvation whereby they mature into Con-vSMLCs. The undifferentiated SMCLs can be prepared by differentiating mammalian pluripotent stem cells (PSCs) by (a) plating a single-cell suspension of PSCs that are smaller than 50 μm at a seeding concentration from about 5×10⁴ cells/cm² to about 1×10⁵ cells/cm² onto a suitable surface; (b) culturing the cells under conditions which prevent the PSCs from aggregating and which induce differentiation of the PSCs into vasculogenic progenitor cells; (c) harvesting the cultured cells and separating them into a single cell suspension of cells that are smaller than 50 μm; and (d) plating the single cell suspension of the harvested cells at a seeding concentration from about 1×10⁴ cells/cm² to about 5×10⁴ cells/cm² on a suitable surface, and culturing the cells in a differentiation medium that is supplemented with platelet-derived growth factor BB (PDGF-BB), a high concentration of serum and transforming growth factor-beta 1 (TGF β1), for a sufficient period of time to allow the vasculogenic progenitor cells to differentiate into SMLCs. In exemplary embodiments of this method, the high concentration of serum is between about 5% and about 20% serum (v/v), for example about 10% serum (v/v). In exemplary embodiments of this method, the serum starvation can include between about 0.5% and 0% serum (v/v). The step of exposing can last, for example, for about six days and the step of treating can last, for example, for about twelve days. In some embdodiments, the step of treating does not include TGF β1. In some embodiments, the conditions that prevent the PSCs from aggregating and induce differentiation of the PSCs into vasculogenic progenitor cells can include culturing the cells on an adhesive substrate, in a differentiation medium that comprises at least about 5% serum (v/v).

In other embodiment, the invention is a method for differentiating undifferentiated vascular smooth muscle-like cells (SMLCs) into vascular smooth muscle-like cells (SMLCs) with a synthetic (Syn-vSMLCs) phenotype in vitro, including the step of exposing the SMLCs to medium that is supplemented with platelet-derived growth factor BB (PDGF-BB), a high concentration of serum and transforming growth factor-beta 1 (TGF β1) to differentiate the SMLCs into Syn-vSMCs. The undifferentiated SMCLs can be prepared by differentiating mammalian pluripotent stem cells (PSCs) by a method that includes the steps of (a) plating a single-cell suspension of PSCs that are smaller than 50 μm at a seeding concentration from about 5×10⁴ cells/cm² to about 1×10⁵ cells/cm² onto a suitable surface; (b) culturing the cells under conditions which prevent the PSCs from aggregating and which induce differentiation of the PSCs into vasculogenic progenitor cells; (c) harvesting the cultured cells and separating them into a single cell suspension of cells that are smaller than 50 μm; and (d) plating the single cell suspension of the harvested cells at a seeding concentration from about 1×10⁴ cells/cm² to about 5×10⁴ cells/cm² on a suitable surface, and culturing the cells in a differentiation medium that is supplemented with platelet-derived growth factor BB (PDGF-BB), a high concentration of serum and transforming growth factor-beta 1 (TGF β1), for a sufficient period of time to allow the vasculogenic progenitor cells to mature into SMLCs. In exemplary embodiments of this method, the high concentration of serum can be is between about 5% and about 20% serum (v/v), for example, about 10% (v/v). The step of exposing can last, fpoor example for about 18 days.

In other embodiments, the invention is a method for differentiating vasculature in vivo by implanting vascular smooth muscle-like cells (SMLCs) differentiated from mammalian pluripotent stem cells (PSCs) that includes the steps of (1) encapsulating SMLCs in extracellular matrix material to form a cell mixture; and (2) injecting the cell mixture subcutaneously into a mammal whereby the SMLCs differentiate into vasculature. The step of encapsulating can further include, for example, combining the cell mixture with additional extracellular matrix material containing an effective amount of basic fibroblast growth factor (bFGF). The effective amount of bFGF can be, for example, about 250 ng/mL bFGF. The step of injecting can use a syringe and needle. In exemplary embodiments of this method, the extracellular material is Matrigel.

The invention is further illustrated by the following nonlimiting examples that show a particular embodiment of the invention. The invention is not intended to be limited to the specific examples shown; it should be understood that this is done for illustration purposes only. Persons skilled in the relevant art will recognize that other components and configurations can be used within the scope of the invention.

### EXAMPLES

### Materials and Methods

Cell Culture

All cells were cultured in humidified incubators, with atmospheres at 37° C. and 5% CO₂.

Human PSCs.

The hESC lines H9 and H13 (passages 15 to 40; WiCell Research Institute, Madison, Wis.) and the hiPSC lines MR31²⁵ and BC1 (28, 29) (kindly provided by Dr. Cheng, JHU School of Medicine) were grown on inactivated mouse embryonic fibroblast feeder layers (GlobalStem, Rockville, Md.) in growth medium comprising 80% ES-DMEM/F12 (GlobalStem), 20% KnockOut Serum Replacement (Invitrogen, Carlsbad, Calif.), and 4 ng/ml basic fibroblast growth factor (bFGF; Invitrogen) or in growth medium composed of KnockOut DMEM (Invitrogen) as basal medium with 20% KnockOut Serum Replacement (Invitrogen), 1% GlutaMAX (Invitrogen), 10 ng/ml FGF2 (PeproTech, Rocky Hill, N.J.), 1% MEM Non-Essential Amino Acids (Invitrogen), 0.1% β-mercaptoethanol (BME; Invitrogen), and 1% antibiotic-antimycotic (Invitrogen). All hPSCs were passaged every four to six days using 1 mg/ml of type IV collagenase (Invitrogen). Media were changed daily.

Human vSMCs.

Human aorta v-SMCs (ATCC, Manassas, Va.; up to passage 7) were used for the control cell type. The cells were cultured according to the manufacturer's recommended protocol in the complete SMC growth medium specified by ATCC, changed media every two to three days, and passaged the cells every three to four days using 0.25% trypsin (Invitrogen). We also examined primary human aorta v-SMCs (Promocell, Heidelberg, Germany; passages 2-5). The cells were cultured following the manufacturer's protocol in their recommended Smooth Muscle Cell Growth Medium 2 (Promocell), changed media every two days, and passaged the cells every three to four days using 0.05% trypsin (Invitrogen).

Vascular SMC Differentiation Protocol.

Human PSCs were collected through digestion with TrypLE (Invitrogen) and a 40 μm mesh strainer (BD Biosciences, San Jose, Calif.) was used to separate the cells into individual cell suspensions. The cells were seeded at a concentration of 5×10⁴ cells/cm² onto plates previously coated with collagen type IV (R&D Systems, Minneapolis, Minn.). The hPSCs were cultured for six days in a differentiation medium composed of alpha-MEM (Invitrogen), 10% FBS (Hyclone), and 0.1 mM β-mercaptoethanol (Invitrogen), with the media changed daily. On day six, the differentiated cells were collected through digestion with TrypLE (Invitrogen), separated with a 40 μm mesh strainer, and seeded at a concentration of 1.25×10⁴ cells/cm² on collagen-type-IV-coated plates. The differentiating hPSCs were cultured in differentiation medium with the addition of 10 ng/ml PDGF-BB (R&D Systems) and 1 ng/ml TGF-β1 (R&D Systems) for six additional days (a total of 12 days) for SMLCs. We cultured hPSC-derived SMLCs for the time periods and with the media components detailed throughout this specification, changing the media every second day. Serum starved cells were passaged every 6-8 days with Tryple, using alpha-MEM (Invitrogen), 10% FBS (Hyclone), and 0.1 mM β-mercaptoethanol (Invitrogen) to neutralize Tryple but then seeded with 0.5% serum media. Because such cells don't proliferate, they should be passaged after certain amount of time.

Real-Time Quantitative RT-PCR.

Two-step reverse transcription polymerase chain reaction (RT-PCR) were performed on differentiated hPSCs at various time points, as described previously (30) Total RNA was extracted using TRIzol (Gibco, Invitrogen), as per the manufacturer's instructions. We verified that all samples were free of DNA contamination. We quantified the concentration of total RNA using an ultraviolet spectrophotometer. RNA (1 μg per sample) was transcribed using the reverse transcriptase M-MLV (Promega Co., Madison, Wis.) and oligo(dT) primers (Promega), following the manufacturer's instructions. The specific assay used was the TaqMan Universal PCR Master Mix and Gene Expression Assay (Applied Biosystems, Foster City, Calif.) for ACTA2, CNN1, SM22, MYH11, COL1, FN1, ELN, MMP1, MMP2, MT1-MMP, SRF, MYOCD, ERK1, YAP1, SMAD3, ACTB, and GAPDH, as per the manufacturer's instructions. The Taqman PCR step was performed with a StepOne Real-Time PCR System (Applied Biosystems), in accordance with the manufacturer's instructions. The relative expressions of the genes was normalized to the amount of β-ACTIN or GAPDH in the same cDNA by using the standard curve method provided by the manufacturer. For each primer set, we used the comparative computerized tomography method (Applied Biosystems) to calculate the amplification differences between the different samples. The values for the experiments were averaged and graphed with standard deviations.

Immunofluorescence.

Cells were fixed using 3.7% formaldehyde fixative for 15 minutes, washed with phosphate-buffered saline (PBS), permeabilized with a solution of 0.1% Triton-X (Sigma-Aldrich, St. Louis, Mo.) for ten minutes, washed with PBS, and incubated for one hour with anti-human α-SMA (1:200; Dako, Glostrup, Denmark), anti-human calponin (1:200; Dako), anti-human SM22 (1:200, Abcam, Cambridge, Mass.), and anti-human SMMHC (3:100; Dako). For ECM staining, cells wer incubated with anti-human fibronectin (1:200; Sigma-Aldrich), anti-human collagen (1:200; Abcam), or anti-human elastin (3:100 Abcam) for one hour. For proliferation, cells were incubated with anti-human Ki67 (1:50, Invitrogen) for one hour. Cells were rinsed twice with PBS and incubated with FITC-conjugated phalloidin (1:40; Molecular Probes, Eugene, Oreg.), anti-mouse IgG Cy3 conjugate (1:50; Sigma-Aldrich), or anti-rabbit IgG Alexa Fluor 488 conjugate (1:1000; Molecular Probes) for one hour. Cells were rinsed with PBS and incubated with DAPI (1:1000; Roche Diagnostics) for ten minutes. Cover slips were rinsed once more with PBS and mounted with fluorescent mounting medium (Dako). The immunolabeled cells were examined using fluorescence microscopy (Olympus BX60; Olympus, Center Valley, Pa.).

Western Blots.

Whole-cell lysates were performed in either a tris-Triton X buffer (1% Triton X, 150 mM NaCl, 50 mM tris, pH 7.5) or in RIPA buffer (150 mM NaCl, 1.0% Triton X, 0.5% sodium deoxycholate, 0.1% SDS, 50 mM tris, pH 8.0) containing 1× protease inhibitor cocktail (Pierce, Rockford, Ill.). We evaluated protein amounts from whole-cell lysates, quantified using the DC assay (BioRad, Hercules, Calif.), and boiled at 95° C. for five minutes in Laemmli buffer (BioRad) with or without BME. We loaded a concentration of 50 μg of isolated protein from each of the indicated samples per well into a 12.5% SDS PAGE gel (BioRad). Proteins were transferred to nitrocellulose membranes, blocked for one hour in 3% nonfat milk, and incubated overnight at 4° C., constantly shaking with primary antibody (antibodies indicated above). Membranes were washed three times in tris buffer saline containing 0.1% Tween-20 (TBST) for 15 minutes each and incubated for two hours at room temperature, constantly shaking with either anti-rabbit HRP (1:1,000; Cell Signaling Technology, Boston, Mass.) or anti-mouse HRP (1:3,000; Cell Signaling Technology). Membranes were washed three times in TBST, developed using enhanced chemiluminescence (Pierce), and visualized using the ChemiDoc XRS+ System (BioRad). Images were acquired using BioRad Quantity One software.

Functional Contraction Studies.

Contraction studies were performed in response to carbachol, as previously described (5, 8, 9, 21, 25, 33). Briefly, hPSC derivatives were cultured (as detailed elsewhere in the paper), washed, and induced for contraction by incubation with 10⁻⁵ M carbachol (Calbiochem, Darmstadt, Germany) in DMEM medium (Invitrogen) for 30 minutes. The cells were visualized using calcein, a cytoplasm-viable fluorescence dye. A series of time-lapse images were taken using a microscope with a 10× objective lens (Axiovert; Carl Zeiss, Thornwood, N.Y.). The cell contraction percentage was calculated as the difference in area covered by the cells before (at time zero) and after contraction (at time 30 minutes). Area analysis was performed with Adobe Photoshop CS5 (Adobe Systems Inc., Mountain View, Calif.), analyzing each set of images three times. Photoshop's magic wand and measurement tools was used to calculate the area of the image not covered in cells, which we then subtracted from the total area of the image. This method improves upon previously established procedure (5, 12) by eliminating the need for image compression and by increasing the consistency of cell selection within each set of images.

Subcutaneous Matrigel Implantation.

PSC-derived vSMCs were trypsinized, collected and stained with PKH26 (Sigma-Aldrich) membrane dye. A total of 0.5×10⁶ PSC-vSMCs was encapsulated in reduced growth factor Matrigel (extracellular matrix material) (BD Biosciences) and 20 μL of EGM-2 media (endothelial growth media). The Matrigel, which contained 250 ng/mL of bFGF (R&D Systems), was loaded, along with the cell mixture, into a 1 mL syringe with a 22-gauge needle and injected subcutaneously into each side of the dorsal region of six- to eight-week-old nude mice. On day 7, isolectin GS-IB4 from Griffonia simplicifolia and Alexa Fluor® 488 conjugate (Invitrogen) was injected through the tail veins of the mice. After 20 minutes, we euthanized the mice by CO₂ asphyxiation and harvested the Matrigel plugs, which were fixed in 3.7% formaldehyde (Sigma-Aldrich) for one hour. A sequence of z-stack images was obtained using confocal microscopy (LSM 510 Meta, Carl Zeiss, Inc.). Vessel diameters from the short axes of the lumen of the vessel were determined from the three-dimensional confocal images. The lumen diameter of vessels that contained areas with and without PSC-vSMC wrapping was measured using ImageJ [National Institutes of Health (NIH)] and known pixel:length ratios. The Johns Hopkins University Institutional Animal Care and Use Committee approved all animal protocols. The investigation conforms to the Guide for the Care and Use of Laboratory Animals published by the US National Institutes of Health (NIH Publication, 8th Edition, 2011).

Histology.

After confocal analysis, the fixed construct explants were dehydrated in graded ethanol (70 to 100%), embedded in paraffin, serially sectioned using a microtome (5 μm), and stained with either hematoxylin and eosin (H&E) or immunohistochemistry for anti-human elastin (Dako, Glostrup, Denmark). Mouse and human tissue samples were used as controls.

Transmission Electron Microscopy (TEM).

Differentiated cells, as detailed below, were prepared for TEM analysis as previously (4 30). Briefly, cultures were fixed with 3.0% formaldehyde, 1.5% in 0.1 M Na cacodylate, 5 mM Ca²⁺, and 2.5% sucrose at room temperature for one hour and washed three times in 0.1 M cacodylate/2.5% sucrose (pH 7.4) for 15 minutes each. The cells were postfixed with Palade's OsO₄ on ice for one hour, rinsed with Kellenberger's uranyl acetate, and then processed conventionally through Epon embedding. Serial sections were cut, mounted onto copper grids, and viewed using a Phillips EM 410 TEM (FEI, Hillsboro, Oreg.). Images were captured using a SIS Megaview III CCD (Lakewood, Colo.).

Statistical Analysis.

All analyses were performed in triplicate for n=3 at least. One Way ANOVA with Bonferroni post-hoc test were performed to determine significance using GraphPad Prism 4.02. (GraphPad Software Inc., La Jolla, Calif.). Significance levels were set at *p<0.05, **p<0.01, and ***p<0.001. All graphical data are reported ±SEM.

Long-Term Culture in High Serum with PDGF-BB and TGFβ1 Induces Synthetic Phenotype

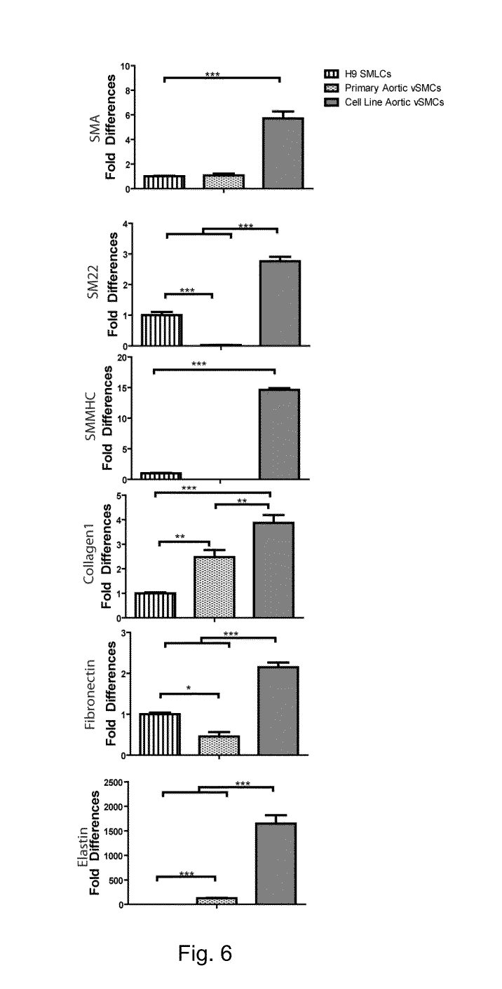

Previous studies established a simple step-wise differentiation protocol, in which hPSCs were differentiated in monolayers supplemented with PDGF-BB and TGF-β1, resulting in highly purified cultures of SMLCs (5, 26) and the parent of the present application. The current study ultimately aimed to mature these SMLCs to contractile phenotype vSMCs. Two principal strategies for the maturation of SMLCs (day 12) were examined: continuous culture in differentiation medium and the effect of deprivation of serum and growth factors during the culture period. The molecular analysis of ECM, cytoskeleton, and contractile proteins enabled the monitoring of the various stages of the maturation process. The aortic vSMC line, which exhibited high expression levels of the contractile proteins, was chosen as the control for mature human vSMCs. These results are shown in FIG. 6.

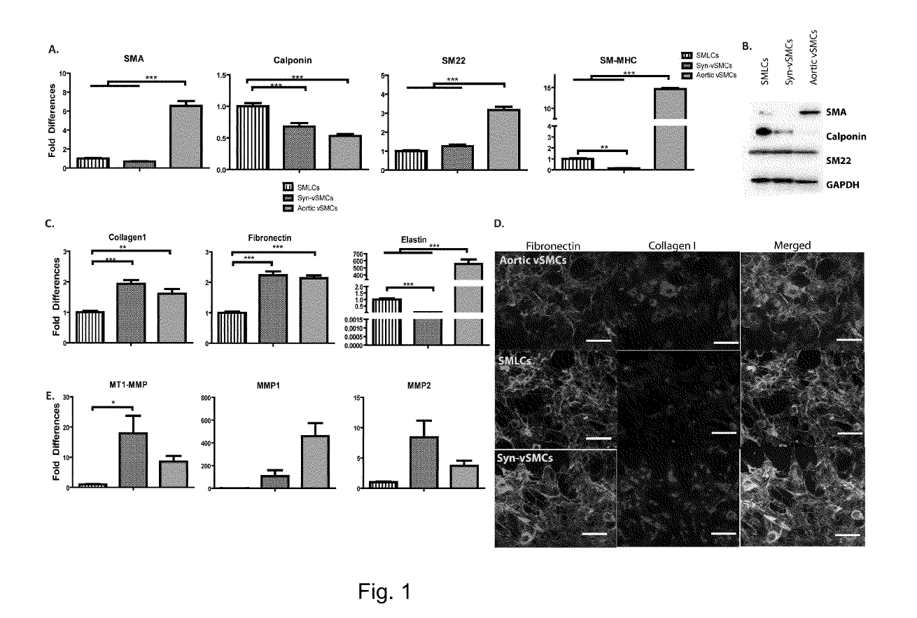

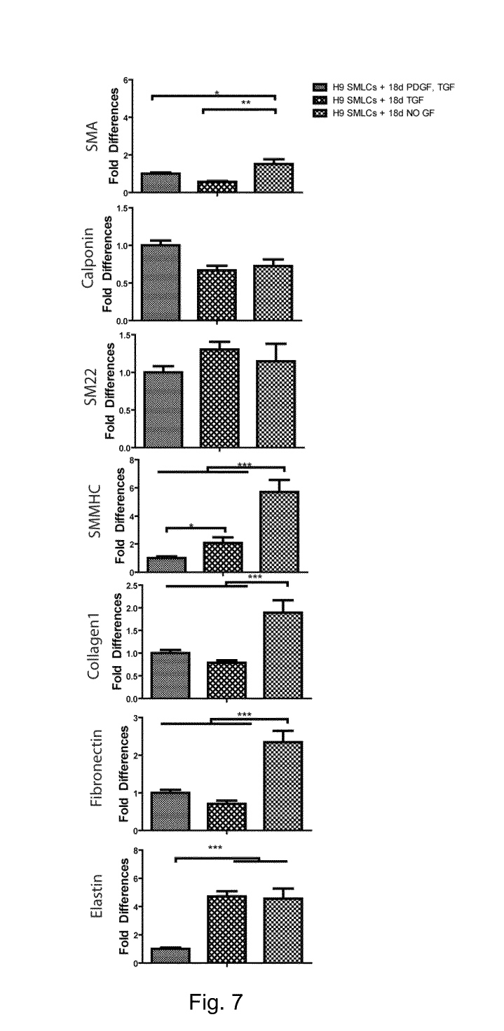

In the first stage, the effect of long-term culture using the differentiation medium was examined. SMLCs (day 12 of differentiation) were cultured for an additional 18 days in differentiation medium containing 10% serum, 10 ng/ml PDGF-BB and 1 ng/ml TGF-β1. Interestingly, the 30-day differentiated cells took on a synthetic vSMC (Syn-vSMC) phenotype compared to SMLCs, including (1) a decrease in calponin mRNA expression, no significant difference in SMA and SM22 mRNA expression, and a decrease in the mRNA expression of SMMHC as is shown in FIGS. 1A and 1B; (2) an increased expression and production of collagen I and fibronectin and a decreased expression of elastin as is shown in FIGS. 1C and 1D; (3) and an increased expression of membrane type 1 matrix metalloproteinase (MT1-MMP)—as well as MMP1 and MMP2 shown in FIG. 1E. These data proved consistent among the different hPSC lines examined. Because it was suggested that PDGF-BB interferes with vSMC maturation (33-36), it was attempted to induce maturation by exposure of day 12 differentiated SMLCs to TGF-β1 or to no growth factors at all. Some detectable increase in the expression of SMMHC and elastin was found, especially when no growth factors were added as is shown in FIG. 7. While these culture conditions did not induce significantly improved contractility, they suggest that a lessening of signaling activation may induce contractile maturation.

### Serum Starvation and PDGF-BB Deprivation Induce Contractile Phenotype

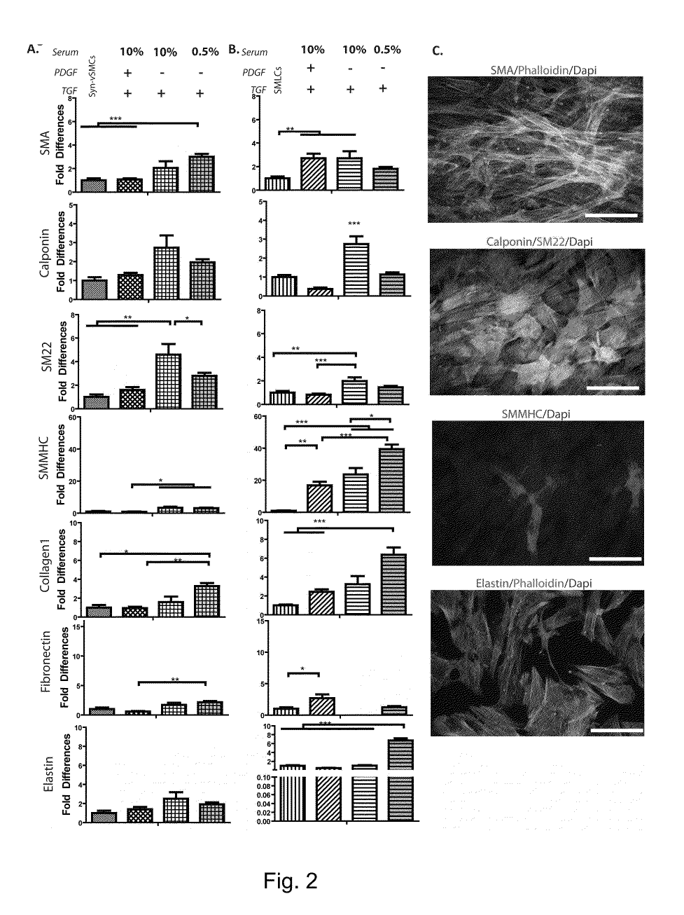

The proposed association of quiescence with the contractile phenotype of vSMCs after birth (31, 32) led to the examination of the effects of serum starvation and growth factor depletion during the differentiation of SMLCs. At first, the Syn-vSMC derivatives were tested for an additional six days in culture in a medium containing 10% serum plus TGF-β1 or 0.5% serum plus TGF-β1. Upregulation in the expression of contractile proteins, specifically SMMHC and elastin, under either of the conditions (see FIG. 2A) was not detected. This implied that Syn-vSMC derivatives had already committed to the synthetic phenotype. Thus, it was attempted to mature the SMLCs (day 12) in the same conditions. Indeed, in medium containing 0.5% serum plus TGF-β1, matured SMLCs (mSMLCs) were detected with significantly upregulated expressions of the contractile proteins SMMHC (approximately fortyfold) and elastin (approximately eightfold) and with no significant change in the expression of early cytoskeleton markers (i.e., αSMA, calponin, and SM22) and ECM proteins (i.e., collagen and fibronectin; as shown in FIG. 2B). The mSMLCs began to acquire a more filamentous cytoskeleton organization, as observed with F-actin, αSMA, calponin, SM22, and occasionally SMMHC; they also began to produce elastin (see FIG. 2C). These data were consistent among the different hPSC lines examined. Culturing the mSMLCs in media containing high concentrations of serum for six days resulted in downregulation of both elastin and SMMHC (data not shown). It should be noted that attempts to differentiate SMLCs in medium without serum (0% serum) could not support cell growth, resulting in extensive cell death after six days. Readding serum to the mSMLCs for another six days resulted in downregulation of SMMHC and elastin (data not shown).

### Long-Term Serum Starvation and TGFβ1 Supplementation Induce Contractile Maturation

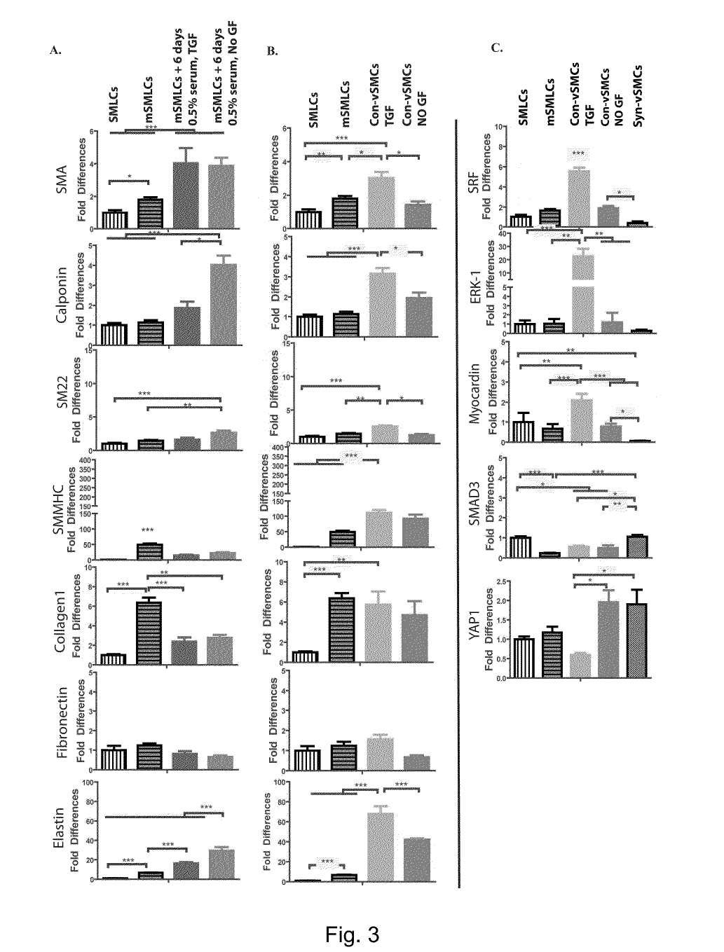

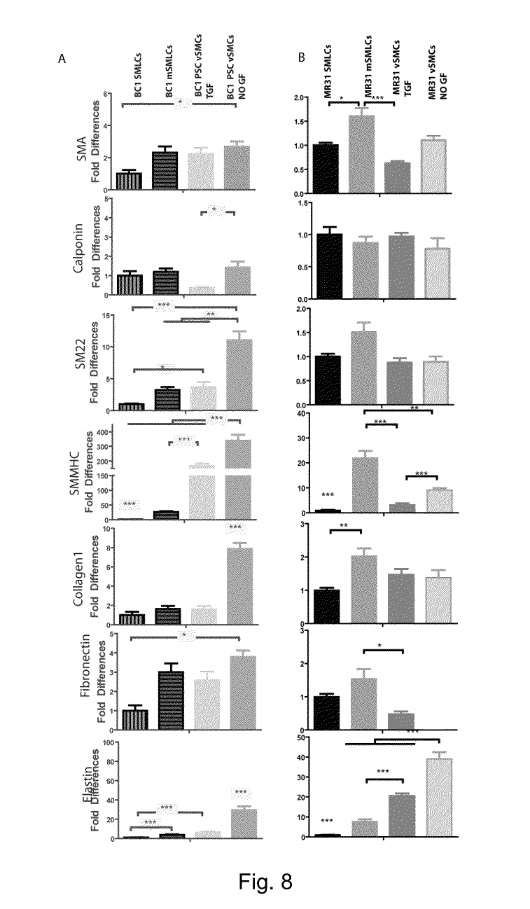

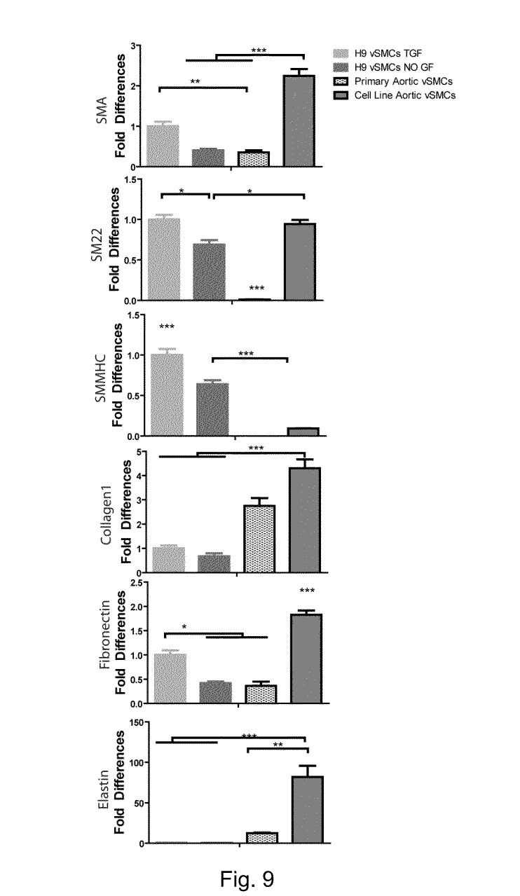

To achieve the maturation of Con-vSMCs from PSCs at levels comparable to those in the body, the effect of short-term (six-day) and long-term (twelve-day) culture in media containing 0.5% serum with and without TGF-β1 was examined. First, as expected, it was noticed that the growth rate decreased along the culture period in low serum. The continuous differentiation of mSMLCs for an additional six days in either set of conditions was not sufficient to induce maturation (as shown in FIG. 3A). Continuous differentiation of the mSMLCs in low-serum medium for 12 days (a total of 30 days of differentiation) induced Con-vSMC maturation, namely the upregulation of SMMHC and elastin, with slightly different responses to the addition of TGF-β1 among different hPSC lines (see FIGS. 3B and 8). Nonetheless, levels of SMMHC expressed in Con-vSMCs were slightly higher than those of aortic vSMCs, while elastin levels were inconsistent in the cell lines (as demonstrated by the large standard deviation) but were, overall, higher than in the Con-vSMCs (shown in FIG. 9). Notably, culturing these Con-vSMCs in low-serum medium with TGF-β1 for up to 18 days maintained high levels of SMMHC and elastin expression with decreasing proliferation rates, whereas culturing them in high-serum medium reduced the levels of SMMHC and elastin expression with increasing proliferation rates (data not shown).

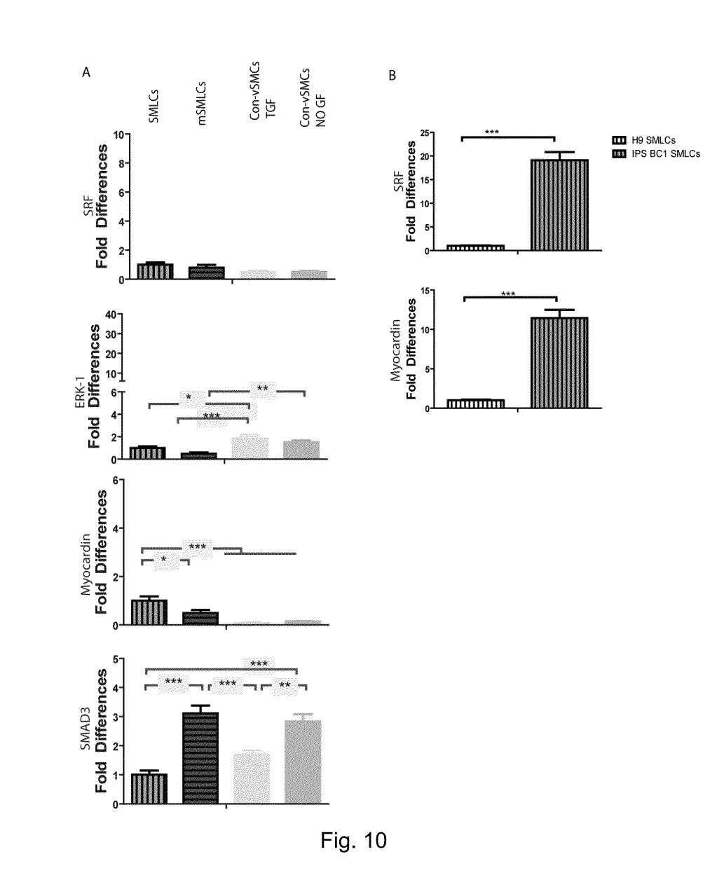

FIG. 3C shows that an upregulation in the expression of myocardin, a serum response factor (SRF) coactivator, through ERK correlates with Con-vSMCs maturation. The activation of the pathway proved more prominent in the hESCs than in the integration-free hiPSCs which can be seen in FIG. 10. Both SMAD3 and YAP1 were upregulated in the Syn-vSMCs compared to Con-vSMCs. Interestingly, these data (FIG. 3C) also suggest that TGF-β1 is imperative for the proper regulation of those pathways.

### Characterization of Syn-vSMC and Con-vSMC Derivatives

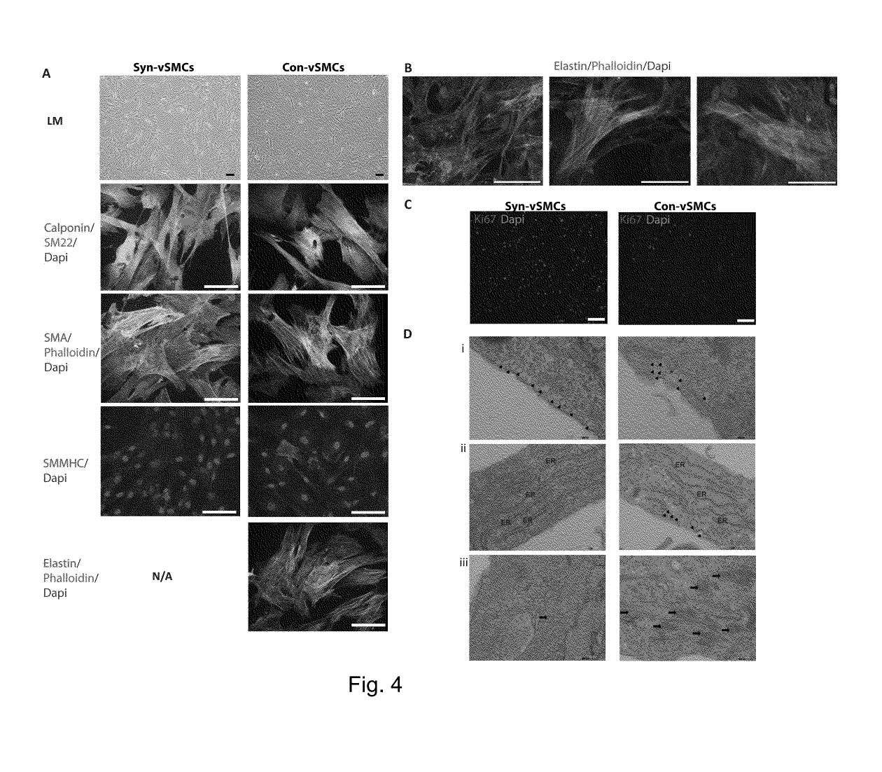

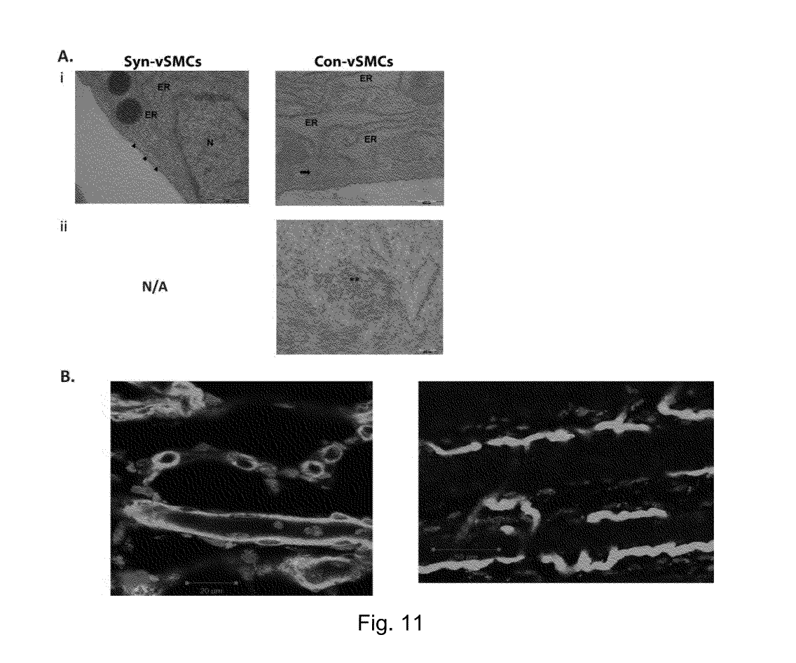

Both Con-vSMCs and Syn-vSMCs are spindle-shaped, with the Syn-vSMCs more elongated as shown in FIG. 4A. Filamentous cytoskeleton organization of F-actin, αSMA, calponin, SM22, and SMMHC was observed in the Con-vSMC while but to a lesser extent in the Syn-vSMCs (FIG. 4A). Accordingly, the hPSC Con-vSMCs had quantitatively more stress fibers per cell (38+/−11) compared to hiPSCs Syn-vSMCs (18+/−3). The hPSC Con-vSMCs displayed larger cell areas (16712.6 μm²+/−11064.0) than hPSC Syn-vSMCs (1825.5 μm²+/−1574.2). Additionally, the hPSC Con-vSMCs also exhibited larger nuclei sizes than the hPSC Syn-vSMCs (468.3+/−183.9 μm vs. 236.6 μm+/−149.6). However, hPSC syn-vSMCs exhibit increased invasion capabilities toward endothelial cells after 48 h compared to hPSC Con-vSMCs. Quantification of the downward invasion in collagen towards endothelial cells revealed that more hPSC Syn-vSMCs invaded the collagen gels compared to hPSCs Con-vSMCs with the average distance traveled by the hPSC syn-vSMCs measured at 451.8 μm compared to 289.3 μm traveled by hiPSC Con-vSMCs. Accordingly, zymography results agreed with the invasion results as Syn-vSMCs expressed both MMP2 and pro-MMP9 while hPSCs Con-vSMCs only expressed pro-MMP2. Migration analysis via a wound healing assay also revealed that the hPSC Syn migrated at a significantly faster average speed compared to iPSC Con-vSMCs (17.1 μm/h+/−7.1 vs 8.6 μm/h+/−3.9). The production of elastin was detected in Con-vSMCs but not in the Syn-vSMCs (FIG. 4A), and the assembly of elastin was further detected after several days in culture (FIG. 4B). Quantification of the Ki67 proliferation marker revealed that Con-vSMCs proliferated slower than Syn-vSMCs (14.15+/−4.20% vs. 83.19+/−10.22%; see FIG. 4C for hESC line; 20.6%+/−9.0 vs. 62.2%+/−3.4 for human iPSC line, data not shown). Finally, TEM analysis revealed that the Con-vSMCs have more (and more active) caveolae than the Syn-vSMCs, which had fewer caveolae as shown in FIGS. 4Di-ii. The Con-vSMC has a larger endoplasmic reticulum (ER) than the Syn-vSMC (see FIG. 4Dii and FIG. 11), while plentiful actin stress fibers (with occasion bundles) were observed in the Con-vSMCs (FIG. 4Diii and FIG. 11).

### Functionality

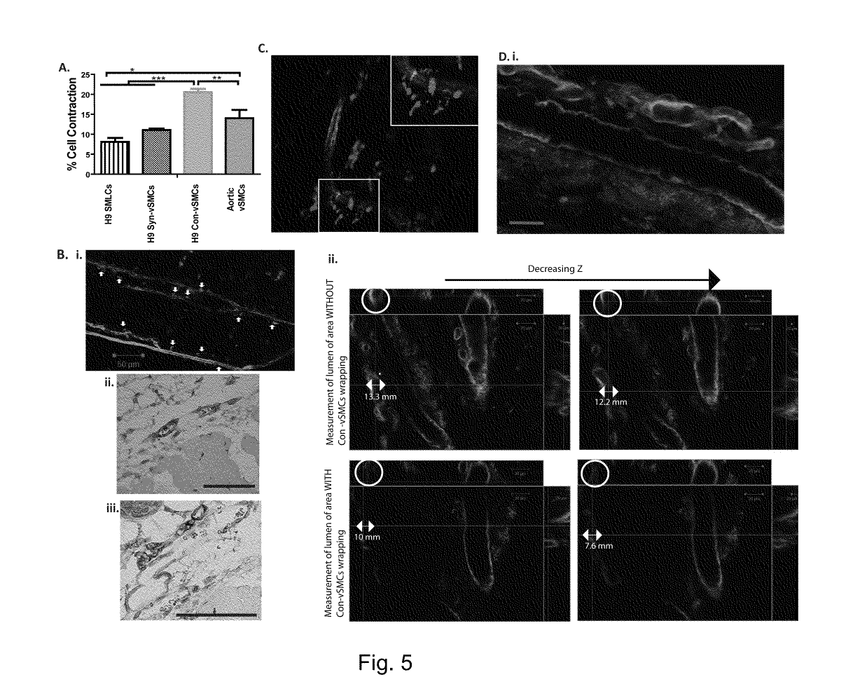

To determine functionality, contractility in vitro was first measured. Contraction studies indicated that Con-vSMCs contract significantly better than Syn-vSMCs; aortic vSMCs with Syn-vSMC contract better than SMLCs; and Con-vSMCs contract similarly to the human aortic vSMC line (see FIG. 5A). Earlier studies demonstrated that vSMC derivatives of human PSCs, which were synthetic by nature, migrate towards and support vasculature both in vitro (5) and in vivo (33). In this case, Syn-vSMC and Con-vSMC interaction with newly formed functional blood vessels was examined. Transplanted Syn-vSMCs and Con-vSMCs were observed migrating to the vasculature and locating in the outer layers of the mouse blood vessels that penetrated into the Matrigel plug (FIG. 5Bi and FIG. 11). In the case of Con-vSMCs, human elastin was further detected around some of the smaller mouse blood vessels that penetrated into the Matrigel plug (FIGS. 5Bii and 5Biii). On some occasions, the human Con-vSMCs were found to wrap the smaller mouse vasculature circumferentially (FIG. 5C), narrowing the endothelial tube (FIG. 5D). These were not observed with the Syn-vSMCs.

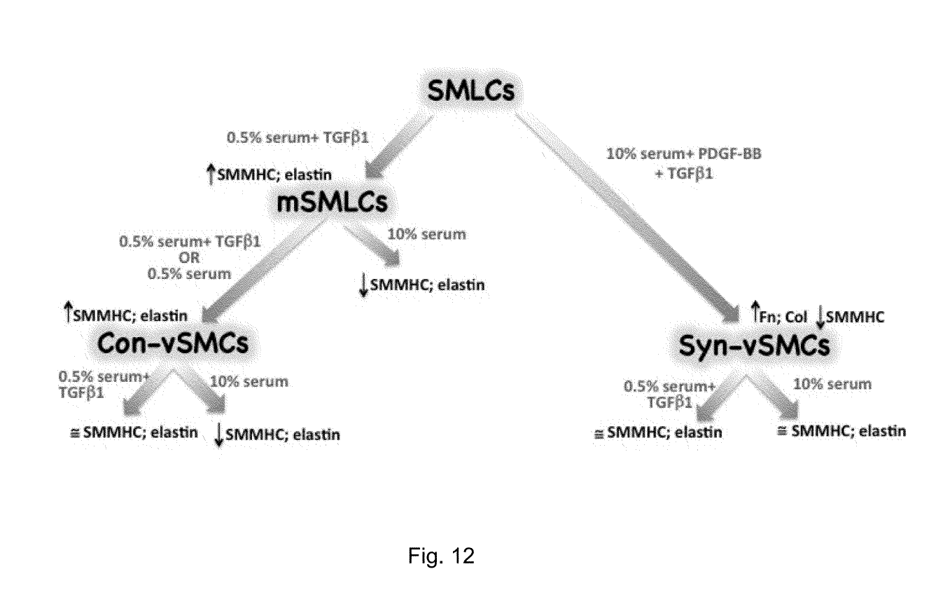

Hence, to achieve the contractile or synthetic maturation of differentiating hPSCs, we use a stage-specific differentiation practice, with appropriate concentrations of factors known to control these developmental steps in the early embryo and in adulthood (see FIG. 12). Moreover, individual hPSC lines require the optimized administration of TGF-β1 for efficient maturation of contractile vSMCs. Such an approach enables the acquisition of the morphological features, cytoskeleton expression, and contractility typical for the contractile phenotype.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above and in the figures are hereby incorporated in their entirety by reference, to the extent permitted by applicable statute or rule, particularly with regard to the method or finding for which they are cited.

### REFERENCES

1. Phelps E A, Garcia A J. Update on therapeutic vascularization strategies. Regen Med 2009; 4:65-80.
2. Gaengel K, Genove G, Armulik A, Betsholtz C. Endothelial-Mural Cell Signaling in Vascular Development and Angiogenesis. Arterioscler Thromb Vasc Biol 2009; 29:630-638.
3. Carmeliet P. Angiogenesis in health and disease. Nat Med 2003; 9:653-660.
4. Hellstrom M, Kal n M, Lindahl P, Abramsson A, Betsholtz C. Role of PDGF-B and PDGFR-beta in recruitment of vascular smooth muscle cells and pericytes during embryonic blood vessel formation in the mouse. Development 1999; 126:3047-3055.
5. Vo E, Hanjaya-Putra D, Zha Y, Kusuma S, Gerecht S. Smooth-Muscle-Like Cells Derived from Human Embryonic Stem Cells Support and Augment Cord-Like Structures In Vitro. Stem Cell Rev 2010; 6:237-247.
6. Grainger D, Metcalfe J, Grace A, Mosedale D. Transforming growth factor-beta dynamically regulates vascular smooth muscle differentiation in vivo. J Cell Sci 1998; 111:2977-2988.
7. Bertolino P, Deckers M, Lebrin F, ten Dijke P. Transforming Growth Factor-β Signal Transduction in Angiogenesis and Vascular Disorders. Chest 2005; 128:585 S-590S.
8. Park S-W, Jun Koh Y, Jeon J, Cho Y-H, Jang M-J, Kang Y et al. Efficient differentiation of human pluripotent stem cells into functional CD34+ progenitor cells by combined modulation of the MEK/ERK and BMP4 signaling pathways. Blood 2010; 116:5762-5772.
9. Schenke-Layland K, Rhodes K E, Angelis E, Butylkova Y, Heydarkhan-Hagvall S, Gekas C et al. Reprogrammed Mouse Fibroblasts Differentiate into Cells of the Cardiovascular and Hematopoietic Lineages. Stem cells 2008; 26:1537-1546.
10. Xu Y, Stenmark K R, Das M, Walchak S J, Ruff L J, Dempsey E C. Pulmonary artery smooth muscle cells from chronically hypoxic neonatal calves retain fetal-like and acquire new growth properties. Am J Physiol Lung Cell Mol Physiol 1997; 273:L234-L245.
11. Dempsey E C, Badesch D B, Dobyns E L, Stenmark K R. Enhanced growth capacity of neonatal pulmonary artery smooth muscle cells in vitro: Dependence on cell size, time from birth, insulin-like growth factor I, and auto-activation of protein Kinase C. J Cell Physiol 1994; 160:469-481.
12. Ball S G, Shuttleworth C A, Kielty C M. Platelet-derived growth factor receptors regulate mesenchymal stem cell fate: implications for neovascularization. Expert Opin Biol Ther 2010; 10:57-71.
13. Owens G K, Kumar M S, Wamhoff B R. Molecular Regulation of Vascular Smooth Muscle Cell Differentiation in Development and Disease. Physiol Rev 2004; 84:767-801.
14. Tuna B G, Bakker E N T P, VanBavel E. Smooth Muscle Biomechanics and Plasticity: Relevance for Vascular Calibre and Remodelling. Basic Clin Pharmacol Toxicol 2012; 110:35-41.
15. Michael S P. Transcriptional programs regulating vascular smooth muscle cell development and differentiation. Curr Top Dev Biol Vol Volume 51: Academic Press:69-89.
16. Yamashita J, Itoh H, Hirashima M, Ogawa M, Nishikawa S, Yurugi T et al. Flk1-positive cells derived from embryonic stem cells serve as vascular progenitors. Nature. 2000; 408:92-96.
17. Hill K L, Obrtlikova P, Alvarez D F, King J A, Keirstead S A, Allred J R et al. Human embryonic stem cell-derived vascular progenitor cells capable of endothelial and smooth muscle cell function. Exp Hematol 2010; 38:246-257.e241.
18. Xiao Q, Zeng L, Zhang Z, Hu Y, Xu Q. Stem cell-derived Sca-1+ progenitors differentiate into smooth muscle cells, which is mediated by collagen IV-integrin α1/β1/αv and PDGF receptor pathways. Am J Physiol Cell Physiol 2007; 292:C342-C352.
19. Huang H, Zhao X, Chen L, Xu C, Yao X, Lu Y et al. Differentiation of human embryonic stem cells into smooth muscle cells in adherent monolayer culture. Biochem Biophys Res Commun 2006; 351:321-327.
20. Sinha S, Hoofnagle M H, Kingston P A, McCanna M E, Owens G K. Transforming growth factor-beta1 signaling contributes to development of smooth muscle cells from embryonic stem cells. Am J Physiol Cell Physiol. 2004; 287:C1560-1568.
21. Vazao H, das Neves R P, Graos M, Ferreira L. Towards the maturation and characterization of smooth muscle cells derived from human embryonic stem cells. PLoS One. 2011; 6:e17771.
22. Jin S, Hansson E M, Tikka S, Lanner F, Sahlgren C, Farnebo F et al. Notch signaling regulates platelet-derived growth factor receptor-beta expression in vascular smooth muscle cell. Circ Res 2008; 102:1483-1491.
23. Oyamada N, Itoh H, Sone M, Yamahara K, Miyashita K, Park K et al. Transplantation of vascular cells derived from human embryonic stem cells contributes to vascular regeneration after stroke in mice. J Transl Med. 2008; 6:54.
24. Taura D, Sone M, Homma K, Oyamada N, Takahashi K, Tamura N et al. Induction and Isolation of Vascular Cells From Human Induced Pluripotent Stem Cells—Brief Report. Arterioscler Thromb Vasc Biol. 2009; 29:1100-1103.
25. Lee T H, Song S H, Kim K L, Yi J Y, Shin G H, Kim J Y et al. Functional Recapitulation of Smooth Muscle Cells Via Induced Pluripotent Stem Cells From Human Aortic Smooth Muscle Cells. Circ Res 2010; 106:120-128.
26. Gerecht-Nir S, Ziskind A, Cohen S, Itskovitz-Eldor J. Human embryonic stem cells as an in vitro model for human vascular development and the induction of vascular differentiation. Lab Invest 2003; 83:1811-1820.
27. Mali P, Chou B K, Yen J, Ye Z, Zou J, Dowey S et al. Butyrate greatly enhances derivation of human induced pluripotent stem cells by promoting epigenetic remodeling and the expression of pluripotency-associated genes. Stem Cells 2010; 28:713-720.
28. Chou B K, Mali P, Huang X, Ye Z, Dowey S N, Resar L M et al. Efficient human iPS cell derivation by a non-integrating plasmid from blood cells with unique epigenetic and gene expression signatures. Cell Res 2011; 21:518-529.
29. Cheng L, Hansen N F, Zhao L, Du Y, Zou C, Donovan F X et al. Low incidence of DNA sequence variation in human induced pluripotent stem cells generated by nonintegrating plasmid expression. Cell Stem Cell 2012; 10:337-344.
30. Hanjaya-Putra D, Bose V, Shen Y I, Yee J, Khetan S, Fox-Talbot K et al. Controlled activation of morphogenesis to generate a functional human microvasculature in a synthetic matrix. Blood 2011; 118:804-815.
31. Chan-Park M B, Shen J Y, Cao Y, Xiong Y, Liu Y, Rayatpisheh S et al. Biomimetic control of vascular smooth muscle cell morphology and phenotype for functional tissue-engineered small-diameter blood vessels. J Biomed Mater Res A 2009; 88A:1104-1121.
32. Beamish J A H P, Kottke-Marchant K, Marchant R E. Molecular regulation of contractile smooth muscle cell phenotype: implications for vascular tissue engineering. Tissue Eng Part B Rev. 2010; 16:467-491.
33. Ferreira L S, Gerecht S, Shieh H F, Watson N, Rupnick M A, Dallabrida S M et al. Vascular Progenitor Cells Isolated From Human Embryonic Stem Cells Give Rise to Endothelial and Smooth Muscle—Like Cells and Form Vascular Networks In Vivo. Circ Res 2007; 101:286-294.
34. Cecchettini A, Rocchiccioli S, Boccardi C, Citti L. Chapter Two—Vascular Smooth-Muscle-Cell Activation: Proteomics Point of View. In: Kwang W J, ed. International Review of Cell and Molecular Biology. Vol Volume 288: Academic Press; 2011:43-99.
35. Lombardi MAR D M, Schwartz S M. Methodologic considerations important in the accurate quantitation of aortic smooth muscle cell replication in the normal rat. Am J Pathol. 1991; 138:441-446.
36. Izzard T D, Taylor C, Birkett S D, Jackson C L, Newby A C. Mechanisms underlying maintenance of smooth muscle cell quiescence in rat aorta: role of the cyclin dependent kinases and their inhibitors. Cardiovasc Res 2002; 53:242-252.
37. Yamamoto M, Yamamoto K, Noumura T. Type I Collagen Promotes Modulation of Cultured Rabbit Arterial Smooth Muscle Cells from a Contractile to a Synthetic Phenotype. Exp Cell Res 1993; 204:121-129.
38. Karnik S K, Brooke B S, Bayes-Genis A, Sorensen L, Wythe J D, Schwartz R S et al. A critical role for elastin signaling in vascular morphogenesis and disease. Development. 2003; 130:411-423.
39. Patel A, Fine B, Sandig M, Mequanint K. Elastin biosynthesis: The missing link in tissue-engineered blood vessels. Cardiovasc Res 2006; 71:40-49.
40. Wang D, Chang P S, Wang Z, Sutherland L, Richardson J A, Small E et al. Activation of cardiac gene expression by myocardin, a transcriptional cofactor for serum response factor. Cell 2001; 105:851-862.
41. Wang Z, Wang D Z, Pipes G C, Olson E N. Myocardin is a master regulator of smooth muscle gene expression. Proc Natl Acad Sci USA 2003; 100:7129-7134.
42. Chen J, Kitchen C M, Streb J W, Miano J M. Myocardin: a component of a molecular switch for smooth muscle differentiation. J Mol Cell Cardiol. 2002; 34:1345-1356.
43. Hoofnagle M H, Neppl R L, Berzin E L, Teg Pipes G C, Olson E N, Wamhoff B W et al. Myocardin is differentially required for the development of smooth muscle cells and cardiomyocytes. Am J Physiol Heart Circ Physiol 2011; 300:H1707-1721.
44. Xie C, Guo Y, Zhu T, Zhang J, Ma P X, Chen Y E. Yap1 Protein Regulates Vascular Smooth Muscle Cell Phenotypic Switch by Interaction with Myocardin. J Biol Chem 2012; 287:14598-14605.
45. Xie W-B, Li Z, Miano J M, Long X, Chen S-Y. Smad3-mediated Myocardin Silencing. J Biol Chem 2011; 286:15050-15057.
46. Thyberg J. Differences in caveolae dynamics in vascular smooth muscle cells of different phenotypes. Lab Invest 2000; 80:915-929.
47. Takahashi K, Tanabe K, Ohnuki M, Narita M, Ichisaka T, Tomoda K et al. Induction of Pluripotent Stem Cells from Adult Human Fibroblasts by Defined Factors. Cell 2007; 131:861-872.
48. Mulvany M J, Aalkjaer C. Structure and function of small arteries. Physiol Rev 1990; 70:921-961.
49. Dingemans K P, Teeling P, Lagendijk J H, Becker A E. Extracellular matrix of the human aortic media: An ultrastructural histochemical and immunohistochemical study of the adult aortic media. Anat Rec 2000; 258:1-14.
50. Wolinsky H, Glagov S. A Lamellar Unit of Aortic Medial Structure and Function in Mammals. Circ Res 1967; 20:99-111.

What is claimed is:

1. A method for differentiating undifferentiated mammalian vascular smooth muscle-like cells (SMLCs) into vascular smooth muscle-like cells (SMLCs) with a contractile (Con-vSMLCs) phenotype in vitro, comprising the steps of:
exposing the SMLCs to serum starvation with TGF β1 to differentiate the SMLCs into mature SMLCs(mSMLCs); and
treating the mSMLCs to continued serum starvation whereby they mature into Con-vSMLCs.

2. The method according to claim 1, wherein the undifferentiated SMCLs are prepared by differentiating mammalian pluripotent stem cells (PSCs) by a method comprising the steps of:
plating a single-cell suspension of PSCs that are smaller than 50 μm at a seeding concentration from about 5×10⁴ cells/cm² to about 1×10⁵ cells/cm² onto a suitable surface;
culturing the cells under conditions which prevent the PSCs from aggregating and which induce differentiation of the PSCs into vasculogenic progenitor cells;
harvesting the cultured cells and separating them into a single cell suspension of cells that are smaller than 50 μm; and
plating the single cell suspension of the harvested cells at a seeding concentration from about 1×10⁴ cells/cm² to about 5×10⁴ cells/cm² on a suitable surface, and culturing the cells in a differentiation medium that is supplemented with platelet-derived growth factor BB (PDGF-BB), a high concentration of serum and transforming growth factor-beta 1 (TGF β1), for a sufficient period of time to allow the vasculogenic progenitor cells to differentiate into SMLCs.

3. The method according to claim 2, wherein the high concentration of serum is between about 5% and about 20% serum (v/v).

4. The method according to claim 2, wherein the high concentration of serum is about 10% serum (v/v).

5. The method according to claim 1, wherein serum starvation is between about 0.5% and 0% serum (v/v).

6. The method according to claim 1, wherein the step of exposing lasts for about six days.

7. The method according to claim 1, wherein the step of treating lasts for about twelve days.

8. The method according to claim 1, wherein the step of treating does not include TGF β1.

9. The method according to claim 2, wherein the conditions in the step of culturing that prevent the PSCs from aggregating and induce differentiation of the PSCs into vasculogenic progenitor cells comprise culturing the cells on an adhesive substrate, in a differentiation medium that comprises at least about 5% serum (v/v).

10. A method for differentiating undifferentiated vascular smooth muscle-like cells (SMLCs) into vascular smooth muscle-like cells (SMLCs) with a synthetic (Syn-vSMLCs) phenotype in vitro, comprising the step of:
exposing the SMLCs to medium that is supplemented with platelet-derived growth factor BB (PDGF-BB), a high concentration of serum and transforming growth factor-beta 1 (TGF β1) to differentiate the SMLCs into Syn-vSMCs.

11. The method according to claim 10, wherein the undifferentiated SMCLs are prepared by differentiating mammalian pluripotent stem cells (PSCs) by a method comprising the steps of:
plating a single-cell suspension of PSCs that are smaller than 50 μm at a seeding concentration from about 5×10⁴ cells/cm² to about 1×10⁵ cells/cm² onto a suitable surface;
culturing the cells under conditions which prevent the PSCs from aggregating and which induce differentiation of the PSCs into vasculogenic progenitor cells;
harvesting the cultured cells and separating them into a single cell suspension of cells that are smaller than 50 μm; and
plating the single cell suspension of the harvested cells at a seeding concentration from about 1×10⁴ cells/cm² to about 5×10⁴ cells/cm² on a suitable surface, and culturing the cells in a differentiation medium that is supplemented with platelet-derived growth factor BB (PDGF-BB), a high concentration of serum and transforming growth factor-beta 1 (TGF β1), for a sufficient period of time to allow the vasculogenic progenitor cells to mature into SMLCs.

12. The method according to claim 10, wherein the high concentration of serum is between about 5% and about 20% serum (v/v).

13. The method according to claim 10, wherein the high concentration of serum is about 10% (v/v).

14. The method according to claim 10, wherein the step of exposing lasts for about 18 days.

15. A method for differentiating vasculature in vivo by implanting vascular smooth muscle-like cells (SMLCs) differentiated from mammalian pluripotent stem cells (PSCs) comprising the steps of:
encapsulating SMLCs in extracellular matrix material to form a cell mixture; and
injecting the cell mixture subcutaneously into a mammal whereby the SMLCs differentiate into vasculature.

16. The method according to claim 15, wherein the step of encapsulating further comprises a step of combining the cell mixture with additional extracellular matrix material containing an effective amount of basic fibroblast growth factor (bFGF).

17. The method according to claim 16, wherein the effective amount of bFGF is about 250 ng/mL bFGF.

18. The method according to claim 15, wherein the step of injecting comprises the use of a syringe and needle.

19. The method according to claim 15, wherein the extracellular material is Matrigel.
